SEQUENCE LISTING (8 sequences)

SEQ ID NO: 1 - protein, 18 aa, Artificial Sequence
  Synthetic peptide Hel 13-5
KLLKLLLKLWLKLLKLLL

SEQ ID NO: 2 - protein, 18 aa, Artificial Sequence
  Synthetic peptide
KLLKLLLKLWKKLLKLLK

SEQ ID NO: 3 - protein, 51 aa, Artificial Sequence
  Synthetic peptide
KKLKKLLKKWKKLLKKLKGGGKKGLLLLLLLLLLLLLLLLLLLLLLLLKKA

SEQ ID NO: 4 - protein, 30 aa, Artificial Sequence
  Synthetic peptide
KKGLLLLLLLLLLLLLLLLLLLLLLLLKKA

SEQ ID NO: 5 - protein, 21 aa, Artificial Sequence
  Synthetic peptide
KLLLLKLLLLKLLLLKLLLLK

SEQ ID NO: 6 - protein, 18 aa, Artificial Sequence
  Synthetic peptide
KKLKKLLKKWKKLLKKLK

SEQ ID NO: 7 - protein, 18 aa, Artificial Sequence
  Synthetic peptide
KLLKLLXXLWLKLXKLLL

SEQ ID NO: 8 - protein, 18 aa, Artificial Sequence
  Synthetic peptide
KLLKLLXXLWXKLXKXLL

(19) United States
(12) Patent Application Publication
(10) Pub. No.: US 2011/0195892 A1
(43) Pub. Date: Aug. 11, 2011

(54) Artifical Pulmonary Surfactant Compositions

(76) Inventors: Sannamu Lee, Fukuoka (JP); Ko Yukitake, Fukuoka (JP); Yoshihiro Nakamura, Fukuoka (JP)

(21) Appl. No.: 11/988,019

(22) PCT Filed: Jun. 28, 2006
(86) PCT No.: PCT/JP2006/313434

§ 371 Date: Feb. 1, 2008

Related U.S. Application Data
(60) Provisional application No. 60/694,701, filed on Jun. 29, 2005.

(51) Int. Cl. A61K 38/17; C07K 7/00; C07K 14/785; A61K 38/04; A61P 11/00

(52) U.S. Cl. 514/1.5; 530/330; 530/329; 530/328; 530/327; 530/326; 530/325; 530/324

(57) ABSTRACT

The peptides according to the present invention have a high surfactant activity, but no or little hemolytic activity and comprises D-amino acid and acids at a rate of approximately 5% to 40% of the structuring amino acids. The pulmonary surfactant composition of the present invention comprises a mixture of the peptide with a natural lecithin such as soy lecithin and so on Therefore, the pulmonary surfactant composition can be expected to be developed as a pulmonary surfactant without using any animal-derived substance, which has a high surfactant activity and can be produced at reasonable costs and on a large scale.

### TECHNICAL FIELD

The present invention relates to an artificial pulmonary surfactant composition. More specifically, the present invention relates to an artificial pulmonary surfactant composition with its surfactant activity improved by introduction of a D-amino acid or acids into its amphiphilic peptide portion. The present invention also relates to a novel peptide which has a surfactant activity but no or little hemolytic activity as well as which is suitable for use as an artificial pulmonary surfactant composition. Moreover, the present invention is concerned with a method for application of such a peptide or artificial pulmonary surfactant composition particularly to serious pulmonary disorders such as RDS, ARDS or the like or other diseases associated with pulmonary surfactants, such as asthma or the like.

### BACKGROUND TECHNOLOGY

Pulmonary surfactants are lipid-protein complexes that are synthesized by and secreted from pulmonary alveoli cells II and govern pulmonary functions playing an essential play for the maintenance of life by decreasing their surface tension (See “Pulmonary Surfactants Now”, edited by Seiichi Yoshida: Shinko Koeki Isho Shuppan, Tokyo, 1990; J. R. Riordan: Molecular Basis of Disease: Pulmonary Surfactant, Ed., Biochem. Biophys. Acta, 77-363, 1998). The deficiency or lack of the pulmonary surfactants causes severe respiratory disorders. Such diseases include, for example, respiratory distress syndrome (RDS) which may occur for newborn babies, particularly immature infants, or acute respiratory distress syndrome (ARDS) which may cause severe respiratory disorders for the adult.

Among the respiratory distress syndromes causing such severe respiratory disorders, the newborn respiratory distress syndromes (RDS) are currently treated by administration of medicine that is an artificial pulmonary surfactant derived from the bovine lung. This medicine, however, is very expensive so that its application to acute respiratory distress syndromes (ARDS) for the adult is currently restricted. Therefore, if pulmonary surfactant preparations could be prepared at reasonably inexpensive costs, it would be applied to such acute respiratory distress syndromes (ARDS) and this can be expected to greatly contribute to the treatment of severe respiratory failures.

Further, recently, there is an increasing necessity for application of pulmonary surfactants not only to RDS and ARDS but also to severe acute respiratory syndromes (SARS) and infectious inflammatory pulmonary diseases such as pneumonia, etc. as well as pulmonary cancers, etc. for mitigation of severe respiratory failure symptoms of the terminal stage, and the pulmonary cancers recently increasing mortality rapidly. Moreover, it is recently known that a pulmonary surfactant substance is secreted from the bronchus so that it is considered to play a role as an expectorant. The pulmonary surfactant is further expected to be useful for many other diseases that require improvements in respiratory failures, including, among others, relieving from a fit of asthma by inhalation of the pulmonary surfactant (Bubu, K. S., et al., Eur Respir J., 21, 1046-1049, 2003). In addition, if less expensive pulmonary surfactants could be developed, they are extremely useful because they could be applied to these diseases.

The pulmonary surfactant is a kind of lipoproteins consisting of a complex between a lipid and a protein as described above. The major component of the lipid is a phospholipid including, for example, dipalmitoylphosphatidylcholine (DPPC), phosphatidylglycerol (PG) and so on. The phospholipids such as DPPC and PG are considered to be an essential component for a surfactant activity. In addition to those lipids, they also include a neutral fat such as cholesterols and triglyceride. On the other hand, the surfactant proteins (SPs) account for approximately 5% of the total composition of the pulmonary surfactant and consist of four kinds, i.e., SP-A, SP-B, SP-C and SP-D. Among these proteins, SP-B and SP-C play an important role in inducing a pulmonary surfactant activity.

A pulmonary surfactant preparation named Surfacten® is commercially available. This pulmonary surfactant preparation consists of 1%-2% proteins (SP-B and SP-C) extracted from bovine lung and a lipid component including DPPC and PG. This commercially available surfactant preparation is effective, however, it is prepared from the bovine lung as a raw material so that it has the problem with safety and expensive expenses. There is a demand, therefore, to develop a surfactant preparation that can sustain safety and that can be prepared at less expensive costs.

It has most recently been reported that “surfaxin” of a completely synthetic type surfactant preparation was designated for priority investigation as an agent for treating bronchopulmonary dysplasia of premature infants by the FDA of the U.S.A. It is also reported that surfaxin was slightly higher in a survival rate than Surfacten® that is an artificial pulmonary surfactant preparation containing a component derived from the bovine lung (Ninha, S. K., et al., Pediatrics, 115, 1030-1038, 2005). In 1991, Cochrane et al. reported for the first time that surfaxin is composed of a mixture of a synthetic peptide KL₄ consisting of 21 amino acids with a certain kind of a lipid (Cochraine, C. G. and Revak, S. D., Science, 254, 566-568, 1991). It is said, however, that surfaxin is estimated to be still very expensive and as nearly expensive as Surfacten®.

The pulmonary surfactant proteins, SP-B and SP-C, are of significance for demonstrating pulmonary surfactant activities, and they have different modes of action against membrane. SP-B is present on the membrane surface, while SP-C is present in the state of penetrating through the membrane. It is therefore considered that they catalyze the mutual migration between the monomolecular membrane and the bimolecular membrane occurring at the pulmonary air-liquid interface.

The present inventors, therefore, come to think about the possibility that an amphiphilic peptide could impart a pulmonary surfactant activity if it could be designed so as to simultaneously have a hydrophilic portion and a hydrophobic portion mimicking the type staying on the membrane surface and the type penetrating through the membrane.

The present inventors have previously found that some peptides could demonstrate surface activities as equal to those of Surfacten®, the peptides being designed on the above concept when used in place of this protein component. These peptides are each composed of L-amino acid in its entirety, and the present inventors et al. have reported that a peptide named He113-5, among others, presented an equal curve to that of Surfacten®, when its surface tension-surface area curve (hysteresis curve) was measured by Wilhelmy tensiometer mimicking the respiratory pressure of the lung (Lee, S., Yukitake, K., Sugihara, G. and Shibata, I.; Japanese Patent Publication No. 2004-305006 A1).

These peptides demonstrate amphiphilic properties which are basic and highly soluble to lipids (Kiyota, T., Lee, S. & Sugihara, G., Biochemistry, 35, 13196-13204, 1996), and further which has the property of penetrating its lipid-soluble portion deeply into an acidic and neutral phospholipid membrane (Kitamura, A. et al., Biophys. J. 76, 1457, 1999). It was found that these peptides in the DPPC-PG-PA (palmitic acid)-peptide mixture systems exhibited better surfactant properties. Further, it is disclosed that these peptides could demonstrate a pulmonary surfactant activity even if one or more amino acids would be lost, substituted or added (Japanese Patent Publication No. 2004-305006 A1). More specifically, it is disclosed that L (leucine) is replaced by another aliphatic hydrophobic residue or K (lysine) is replaced by another aliphatic hydrophilic residue as well as W (tryptophan) is replaced by another aliphatic hydrophobic residue or an aromatic hydrophobic residue.

Moreover, the present inventors indicated the possibility of using an inexpensive soy lipid in place of expensive DPPC and PG (PCT/JP2005/8234; Inventors: S. Lee, K. Yukitake & Y. Nakamura). The surfactant contains L-α-phosphatidylcholine (PC) in an amount as high as approximately 60% and a half of the amount comprises DPPC of a saturated lipid type. The high content of this DPPC is said to be one factor that can prevent a collapse of the lung. Therefore, the present inventors have used, as a substitute for DPPC composed of the saturated lipid, a fractionated lecithin of a high PC content, which is selected out of hydrogenated soy lecithin obtained by hydrogenation of soy lecithin. As a result, it was found that the system of hydrogenated soy lecithin-soy lecithin-palmitic acid-peptide demonstrated properties as high as Surfacten®, a commercially available surfactant preparation. It is to be noted herein that, concerning the use of soy lecithin, there is a report of a lipid mixture system in which a soy lecithin system was added in a small amount as an unsaturated lipid system while DPPC-DSPC (distearoyl PC) of the saturated lipid system was used as a major system, but there is no report regarding hydrogenated lecithin (Japanese Patent Publication No. S62-96425 A1).

It is further to be noted herein that the peptide itself has a strong hemolytic property when used singly (Kiyota, T., Lee, S. & Sugihara, G., Biochemistry, 35, 13196-13204, 1996). These peptides, however, demonstrate no hemolytic property when used as a complex with the lipid so that they are considered to cause little practical problem even if they were used as a medicine. Nevertheless, it is useful in terms of further heightening safety of medicine that a peptide having no hemolytic activity is used.

In addition, recently, it has been reported that the introduction of D-amino adds to a basic amphiphilic peptide showing a hemolytic activity can produce specificity and selectivity reducing a hemolytic activity (Papo et al., J. Biol. Chem., 278, 210-18, 2003).

### SUMMARY OF INVENTION

Therefore, as a result of review, it was found by the present inventors that a novel surfactant preparation, has a higher activity than Surfacten®, a commercial available medicine, the surfactant preparation containing an amphiphilic peptide previously synthesized to provide it with specificity to a mode of action against membrane, in which a portion of the structuring amino acids is replaced by D-amino acid or acids, and containing a soy lecithin which is inexpensive soy lipid, such as hydrogenated soy lecithin, used in place of expensive DPPC and PG. The present invention was completed on the basis of this finding (U.S. Patent Application Ser. No. 60/694,701; Inventors: Lee, S., Yukitake, K. & Nakamura, Y.).

Therefore, the present invention has the object to provide an amphiphilic peptide composed of a hydrophilic portion and a hydrophobic portion but having no or little hemolytic activity.

The present invention in another embodiment has the object to provide an artificial pulmonary surfactant containing the above peptide and a natural lipid.

The present invention in its further embodiment has the objects to provide a method for using the above peptide for the artificial pulmonary surfactant and to provide a method for using the peptide or the artificial pulmonary surfactant composition for treating diseases associated with a pulmonary surfactant.

In order to achieve the objects as described above, the present invention provides the amphiphilic peptide having a hydrophilic portion and a hydrophobic portion, which is composed of an amino acid sequence consisting of from approximately 5 to 60 amino adds, preferably from approximately 10 to 40 amino acids, more preferably from approximately 10 to 20 amino adds, and which has a surfactant activity but no hemolytic activity.

More specifically, the present invention provides a peptide composed of L-acids with D-amino acid or acids accounting for from approximately 5% to 60%, preferably from approximately 10% to 40%, more preferably from approximately 20% to 30%, of the amino acid sequence of the structuring amino acids. In other words, the peptide is composed of from one D-amino acid to plural amino acids, preferably from one to ten amino adds, more preferably from one to seven amino acids, particularly preferably from two to six amino acids.

In a more preferred embodiment, the peptide according to the present invention contains lysine and/or leucine as the major structuring amino acid. In another preferred embodiment, the peptide with L-lysine and/or L-leucine replaced by D-lysine and/or D-leucine, respectively, is provided. In another preferred embodiment, the peptide containing tryptophan in addition to lysine and/or leucine is provided.

Moreover, the present invention provides an artificial pulmonary surfactant composed of the above peptide and a natural lipid, generally floral lipid, preferably soybean lipid.

In addition, the present invention in another embodiment provides a method for using the above peptide for an artificial pulmonary surfactant composition and to provide a method for using the artificial pulmonary surfactant composition for treating diseases associated with a pulmonary surfactant.

### BRIEF DESCRIPTION OF THE ACCOMPANYING DRAWINGS

FIG. 1 is a graph showing CD and HPLC data of a peptide containing peptide Hel 13-5 and D-amino acids.

FIG. 2 is a graph showing a hysteresis curve of peptides Hel 13-5D3 and Hel 13-5D5 each containing D-amino adds.

FIG. 3 is a graph showing lung recovery effects of various kinds of pulmonary surfactants using lung-irrigating model rats.

FIG. 4 is a graph showing the effects of pulmonary surfactants in asthma models.

### BEST MODE FOR CARRYING OUT THE INVENTION

The peptide according to the present invention comprises an amphiphilic peptide having a hydrophilic portion and a hydrophobic portion in its moiety, which is provided with specificity to a mode of action to membrane and which has no or little hemolytic action. The terms “amphiphilic peptide” used herein are intended to mean a peptide which demonstrates an amphiphilic property having a basic property and a high solubility in lipid as well as which permits its lipid-soluble portion to penetrate deeply into an acidic and neutral phospholipid membrane. The peptide may include a natural peptide or a synthetic peptide as long as it is provided with such properties. Further, the amphiphilic peptide may be the one having ten or more hydrophobic amino acid residues in its moiety and such hydrophobic amino acid residues may be of one kind or two or more kinds. Moreover, the amino acid residues may include, for example, leucine, lysine and so on and further comprise tryptophan.

The peptide according to the present invention may be an amphiphilic peptide which is composed of from approximately 5 to 60 amino adds, preferably from approximately 10 to 40 amino acids, more preferably from approximately 10 to 20 amino adds and comprises lysine and/or leucine as the major structuring amino acid, and contains some D-amino acid or acids replaced into the place of L-amino acid or acids of the original peptide. The number of the D-amino acids to be replaced may account for from approximately 5% to 50%, preferably from approximately 10% to 40%, more preferably from approximately 20% to 30% of the total amino acid sequence. In other words, the number of the D-amino acids may be one to ten amino acids, preferably one to seven amino acids, more preferably from two to six amino acids, although it may depend upon the total number of the amino acids constituting the peptide.

An example of the peptide according to the present invention is illustrated as a sequence listing as described hereinafter. In the below-described illustration, L-lysine and/or L-leucine are/is replaced by D-lysine and/or D-leucine, respectively. It is to be understood, however, that the peptide of the present invention is not limited to those as described herein and the amino acid to be replaced may be selected appropriately and replaced by an appropriate amino acid or acids. It is to be noted herein, however, that the present invention is not limited at all to those peptides as described herein.

For example, the peptides (Sequence ID #1 to #6) as will be described hereinafter are disclosed in Japanese Patent Publication No. 2004-305,006 A1, and they may be used for the present invention by replacing leucine and lysine of the below peptide by D-leucine and D-lysine, respectively.

Peptide Hel 13-5
(Sequence ID #1)
NH₂-Lys Leu Leu Lys Leu Leu Leu Lys Leu Trp Leu
Lys Leu Leu Lys Leu Leu Leu-COOH
Peptide Hel 11-7
(Sequence ID #2)
NH₂-Lys Leu Leu Lys Leu Leu Leu Lys Leu Trp Lys
Lys Leu Leu Lys Leu Leu Lys-COOH
Peptide Hel 17-11-P24
(Sequence ID # 3)
AcNH-Lys Lys Leu Lys Lys Leu Leu Lys Lys Trp Lys
Lys Leu Leu Lys Lys Leu Lys Gly Gly Gly Lys Lys
Gly Leu Leu Leu Leu Leu Leu Leu Leu Leu Leu Leu
Leu Leu Leu Leu Leu Leu Leu Leu Leu Leu Leu Leu
Leu Lys Lys Ala-CONH₂
Peptide P24
(Sequence ID #4)
AcNH-Lys Lys Gly Leu Leu Leu Leu Leu Leu Leu Leu
Leu Leu Leu Leu Leu Leu Leu Leu Leu Leu Leu Leu
Leu Leu Leu Leu Lys Lys Ala-CONH₂
Peptide KL24
(Sequence ID #5)
NH₂-Lys Leu Leu Leu Leu Lys Leu Leu Leu Leu Lys
Leu Leu Leu Leu Lys Leu Leu Leu Leu
Lys-COOH
Peptide Hel 17-11
(Sequence ID #6)
NH₂-Lys Lys Leu Lys Lys Leu Leu Lys Lys Trp Lys
Lys Leu Leu Lys Lys Leu Lys-COOH

In addition, the peptide according to the present invention may include, for example, peptide Hel 13-5D3 (Sequence ID#7) in which leucine at the positions 7 and 14 as well as lysine at the position 8 of the peptide Hel 13-5 (Sequence ID#1) are replaced by D-leucine as well as D-lysine, respectively, and peptide Hel 13-5D5 (Sequence ID#8) in which leucine at the positions 7, 11, 14 and 16 as well as lysine at the position 8 of the peptide Hel 13-5 (Sequence ID#1) are replaced by D-leucine as well as D-lysine, respectively.

Peptide Hel 13-5D3
(Sequence ID #7)
NH₂-Lys Leu Leu Lys Leu Leu D-Leu D-Lys Leu Trp
Leu Lys D-Leu Leu Lys Leu Leu
Leu-COOH
Peptide Hel 13-5D5
(Sequence ID #8)
NH₂-Lys Leu Leu Lys Leu Leu D-Leu D-Lys Leu Trp
D-Leu Lys Leu D-Leu Lys D-Leu
Leu Leu-COOH

Among the peptides according to the present invention, the synthetic peptides may be prepared by means of chemical techniques as well known to the art involved.

The chemical techniques may include, for example, peptide synthesis techniques by means of usual liquid phase method or solid phase method. More specifically, such peptide synthesis methods may comprise, for example, a solid phase method including, for example, Fmoc chemistry method and Boc-chemistry method, in which each amino acid is conjugated one by one on a resin on the basis of information on an amino acid sequence, thereby extending its chain to a target peptide, and a liquid phase method including, for example, fragment condensation method in which fragments composed of several amino acids are synthesized in advance and the fragments are coupled with one another.

As the condensation methods capable of being adopted for the peptide synthesis, there may be used various kinds of methods well known to the art, and the methods may specifically include, for example, benzoyltriazole-related condensation method (e.g., HATU, TBTU, etc.), DCC method, active ester method and so on. As a solvent to be used for each of these methods, there may be used a general type of solvents that can be used for such peptide condensation methods and they may include, for example, dimethylformamide (DMF), dimethylsulfoxide (DMSO), hexaphosphoroamide, dioxane, tetrahydrofuran (THF), ethyl acetate and so on and a mixture thereof.

In the case of the peptide synthesis reaction, the carboxyl groups of the amino acids and the peptides, which are not involved with the reaction, are not necessarily required to be protected as long as they do not affect the reaction adversely, however, they may generally be protected, for example, by esterification forming a lower alkyl ester such as tertiary butyl ester or the like, benzyl ester, p-methoxybenzyl ester, p-nitrobenzyl ester, p-phenacyl ester or the like. An amino acid, lysine, having a functional group at a side chain may be protected by a protective group such as benzyloxycarbonyl group, tert-butyloxycarbonyl group or the like, while tryptophan residue may not be protected. Further, these protective groups may be removed readily in accordance with conventional methods using, for example, piperidine, contact reduction, hydrogen chloride, trifluoroacetic acid, methane sulfonic acid and so on.

As the peptide of the present invention contains the D-amino acid or adds, the synthesis of the peptide by genetic engineering method may be faced with difficulties so that the genetic engineering method cannot be generally said to be very effective, although it can be utilized for the synthesis of some kinds of peptides.

The peptide of the present invention can be appropriately purified by methods which have been usually and extensively used in the peptide chemistry field, including, for example, ion exchange resin chromatography, distribution chromatography, gel chromatography, affinity chromatography, high-performance liquid chromatography (HPLC), and so on.

The artificial pulmonary surfactant composition according to the present invention comprises a mixture of the peptide with a phospholipid as a natural lipid, including, but being not limited to, soy lecithin, egg yolk lecithin, and so on. The artificial pulmonary surfactant may contain a saturated higher alcohol including, but being not limited to, octadecanol and so on, a fatty acid, and a neutral fat, such as cholesterol, triacyl glycerol and so on. Among those components, the fatty acid may include, but not be limited to, a free fatty acid, an alkali metal salt of a fatty acid, an alkyl ester of a fatty acid, a glycerin ester of a fatty acid or an amide of a fatty acid or a mixture of two kinds or more. The free fatty acid may, but not be limited to, palmitic cid (PA), myristic acid, stearic acid and so on.

Further, the artificial pulmonary surfactant may contain other phospholipids, in addition to the above phospholipids, which may include, but not be limited to, a 1,2-diacylglycero-(3)-phosphocholine such as 1,2-dipalmitoylglycero-(3)-phosphocholine (dipalmitoylphosphatidylcholine (DPPC)), 1,2-distearoylglycero-(3)-phosphocholine, 1-palmitoyl-2-stearoylglycero-(3)-phosphocholine and 1-stearoyl-2-palmitoyl-glycero-(3)-phosphocholine, a 1-alkyl-2-acylglycero-(3)-phosphocholine such as 1-hexadecyl-2-palmitoylglycero-(3)-phosphocholine and 1-octadecyl-2-palmitoylglycero-(3)-phosphocholine, a 1,2-dialkyl-glycero-(3)-phosphocholine such as 1,2-dihexadecyl-glycero-(3)-phosphocholine, a phosphatidylethanol amine such as dioleylphosphatidylethanol amine (DOPE), a 1,2-diacyl-sn-glycero-(3)-phosphoric acid (L-α-phosphatidic acid), a 1,2-diacyl-sn-glycero-(3)-phospho-L-serine (phosphatidylserine), a 1,2-diacyl-sn-glycero-(3)-phospho-sn-glycerol (phosphatidylglycerol (PG)), diphosphatidylglycerol, a 1,2-diacyl-sn-glycero-(3)-phospho-(1)-L-myo-inositol (phosphatidylinositol), and so on.

The amount of the peptide in the artificial pulmonary surfactant composition may be appropriately decided depending upon the kind of the peptide and the lipid, although it is not limited to a particular one. In accordance with the present invention, a ratio of the peptide to the lipid may be in the range, for example, of from 1% to 70% (w/w).

The present invention will be described be in more detail by examples, but it should be understood that the present invention is not interpreted as being limited in any respect to the examples which will be described hereinafter.

### EXAMPLES

### Example 1

### (Synthesis of Peptide)

The synthesis of the peptide was carried out by using a Fmoc-Leu-PEG resin (Watanabe Kagaku K.K.: Fmoc-Leu-OH, 0.21 mmol/g) as a starting material by a continuous flow-type Fmoc solid phase synthesis method with an automatic synthesis machine (PerSeptive Biosystems). The protective group of the resin was eliminated from the resin with trifluoroacetic acid, and the resulting crude peptide was dissolved in 30% acetic acid, followed by collecting the peptide portion by Sephadex G-25 column chromatography. The collected peptide portion was then purified by tertiary water (acetonitrile type) containing 0.1% TFA by a reverse-phase liquid chromatography (HPLC) (COSMOSIL 5C18-AR20×250 mm).

The objective peptide was confirmed by an HPLC analysis (COSMOSIL 5C18-AR4.6×150 mm) using a solvent system (a tertiary water of acetonitrile type containing 0.1% TFA) with a TOF-Mass analysis method (Voyager Model; PerSeptive Biosystems).

As a result, it was confirmed that leucine at the positions 7 and 14 as well as lysine at the position 8 of the peptide Hel 13-5 were replaced by D-leucine and D-lysine, respectively, to form the peptide Hel 13-5D3 (Sequence ID #7) as well as leucine at the positions 7, 11, 14 and 16 and lysine at the position 8 of the peptide Hel 13-5 were replaced by D-leucine and D-lysine, respectively, to form the peptide Hel 13-5D5 (Sequence ID #8).

### (Measurement for CD Spectrum)

The measurement for a CD spectrum was carried out with a spectrometer (Nippon Bunko; 3-700 Spectrometer). The measurement was conducted four times at 25° C. in a wavelength range of 196-260 nm by using a 0.1 cm-long quartz cell (with a water jacket).

The peptide solution (ca. 2 mg) was dissolved in 2 ml of 20 mM Tes Buffer (with 150 mM NaCl), and the concentration of Trp (molar absorbency index: 5,000/cm mol) was determined at an absorbency of 280 nm.

### Example 2

### (Preparation of Lipid Materials and Samples (Lipids or Peptide-Lipid Mixtures))

As the phospholipid, there was used L-α-phosphatidylcholine (egg PC: Avanti Polar Lipids, Inc.) purified from egg yolk. As hydrogenated soy lecithin, there was used hydrogenated soy lecithin (SIP while H; Tsuji Seiyu K.K., Japan), and as fractionated soy lecithin, there was used fractionated lecithin (SLP-PC70; Tsuji Seiyu K.K., Japan). Further, there was used lecithin (soy lecithin PC 70D: Dojin Kagaku K.K., Japan) prepared by further fractionating soy lecithin PC70 colored in yellow brown to remove almost all of the color ingredients. As egg yolk lecithin and other lipids as well as reagents, there were used those produced by Wako Jyunyaku K.K. (Japan). Surfacten® produced by Mitsubishi Weipharma K.K. (Japan) was used, as well as Exosurf and Surfaxin were prepared in accordance with specifications as disclosed in publication.

The peptide, lipid, fatty acid and alcohol were weighed each to a given amount, and each of them was dissolved in a chloroform/methanol mixture. To each sample was added the peptide to reach a 2.5% concentration (w/w), and nitrogen gas was blown into the resulting peptide-lipid mixture. Then, the mixture was dried under reduced pressure to evaporate the organic solvent thoroughly, forming a film-shaped dry product on the wall surface of the container. To this was added physiological saline, and the solution was stirred to form a suspension that was in turn used as a sample.

The composition of each sample is as follows:
Sample A: octadecanol-egg PC-PA (40:35:25 w/w)
Sample B: fractionated soy lecithin 70D-hydrogenated soy lecithin-PA (40:20:20)
Sample C: OD-egg PC-PA-Hel 13-5 (40:35:22.5:2.5)
Sample D: fractionated soy lecithin 70D-hydrogenated soy lecithin-PA-Hel 13-5 (40:40:17.5:2.5)
Sample E: fractionated soy lecithin 70D-hydrogenated soy lecithin-PA-Hel 13-5D3 (40:40:17.5:2.5)
Sample F: Murosurf SLPD5; fractionated soy lecithin 70D-hydrogenated soy lecithin-PA-Hel 13-5D5 (40:40:17.5:2.5)

### (Procedures of Surface Tension Test)

The surface tension was measured at room temperature (25° C.) with Wilhelmy Balancer (Acoma Ika Kogyo K.K.). A Teflon® water bath (78×138×30 mm) was filled with physiological saline to form a dosed liquid surface, and each of the above samples was developed by 100 μg on the air-liquid interface of the liquid surface, allowing the sample to stand for three minutes until the sample spread spontaneously. During this period of time, a variation in the surface tension was recorded as a surface spreading rate using a platinum plate hanging perpendicularly in the water bath. The monomolecular membrane formed in three minutes repeated compressing and expanding its surface area from a range of from 45 cm² at the maximum to 9 cm² at the minimum at a cycle of three minutes. The surface tension acted onto the platinum plate was converted into electrical signals with a power converter and automatically recorded continuously with an X-Y recorder, together with the variation in the surface tension. This measurement was continued until the variation could not be recognized any longer.

### (Assessment of Activity of Pulmonary Surfactants by Animal Tests)

A rat lung was irrigated with warm physiological saline to form a pulmonary surfactant-deficient model rat, and the model rat was allowed to stand in 100% oxygen by artificial ventilation in order to review the effects of the artificial pulmonary surfactants of the present invention on extension of life and pulmonary functions by measuring the pulmonary compliance. As a control, there were used three kinds of surfactants, i.e., Surfacten® derived from bovine pulmonary surfactant, which has been extensively applied clinically, Surfaxin (peptide KL24 (Sequence ID #5): KL24) composed of lysine (K) and leucine (L) and containing DPPC as a major component, and Exosurf® containing no peptide and composed of the lipid system only. Moreover, the case where no pulmonary surfactant was administered was also used as a control. It is to be noted here that the lung irrigation was carried out until the initial pulmonary compliance of 0.60 ml/cmH₂O reached 0.2 ml/cmH₂O. Further, each kind of the surfactants was administered to the pulmonary surfactant-deficient model rats after confirmation of the formation of the pulmonary surfactant-deficient model rats. Each surfactant was administered to a group of six model rats.

### (Effects of Pulmonary Surfactants on Asthma Model)

Brown-Norway rats were sensitized with egg white albumin (OVA) to form asthma model rats that could raise pulmonary resistance by inhalation of OVA. Before inhalation of OVA, the model rats were pre-treated by administering each of the samples, i.e., fractionated soy lecithin 70D-hydrogenated soy lecithin-PA, fractionated soy lecithin 70D-hydrogenated soy lecithin-PA-Hel 13-5, fractionated soy lecithin 70D-hydrogenated soy lecithin-PA-Hel 13-5D3 and Surfacten® in an amount of 0.1 ml (20 mg/ml) into the airway, and OVA was inhaled. The lung resistance was measured periodically in accordance with the Giles et al. method and the effects on asthma were determined by the action of a control over the enhancement of the pulmonary resistance. The statistical analysis was made regarding significance of each group by Tukey-Kremer method (p<0.05).

### (Hemolytic Activity)

The hemolytic activity of each peptide was measured in the way as will be described hereinafter.

Each peptide (ca. 1 mg) was dissolved in 20 (l of 100% acetic acid and diluted with phosphate buffer to reach a total amount of 5 ml. The resulting solution was measured for its absorbency (280 nm) to determine its concentration. The solution was then used for experiments in situ by dilution with phosphate buffer.

A blood sample (ca. 3 ml) was centrifuged at 2,000 rpm at 4° C. for 10 minutes and the supernatant was removed to form an erythrocyte sample. To the erythrocyte sample was added 1 ml of phosphate buffer, and the resulting solution was stirred well, followed by centrifugation in the manner as described above. These procedures were repeated three times to obtain the erythrocyte alone. Moreover, 1 ml of phosphate buffer was added to the resulting erythrocyte, and the resulting mixture was stirred well, followed by distributing it by 60 μl to microtubes and centrifuging to remove the supernatant therefrom. The resulting erythrocyte was in turn used for further experiments.

The peptide and a protein solution were then added by 1 ml to the erythrocyte sample, and the resulting solution was stirred well, followed by allowing it to stand at 25° C. for 60 minutes. Thereafter, the tubes were centrifuged to collect the supernatant only (100 μl) which was in turn measured at an absorbency of 542 nm. The supernatant was then returned to each tube and a drop of surfactant Triton® X was added to each tube, followed by stirring it well and measuring for its absorbency at 542 nm.

As a result, it was confirmed that the hemolytic activity of the peptide, Hel 13-5D3, was concentration dependent and the hemolytic level reached nearby 100% at approximately 20 μM, while the peptide Hel 13-5D5 increased the hemolytic activity to the maximum level of 35% at 20-50 μM and in a concentration-dependent fashion at concentrations ranging from 50 μM to 70 μM. On the other hand, Hel 13-5 was completed hemolyzed at 1 to 5 μM.

(Designing D-Amino Acid-Containing Peptides with High Specificity to Membrane)

In the two-dimensional structure of a natural protein and peptide, the amphiphilic structure composed of its hydrophilic portion and hydrophobic portion has a characteristic hydrophobic-hydrophilic balance (hereinafter referred to as “HHB”) depending upon its structuring amino acids. A difference in the HHB is of great significance in order to determine the stereochemistry, stability and physiological functions of the protein and peptide. Recently, it was reported that the partial introduction of D-amino acid or acids into an amphiphilic peptide caused a subtle variation in the two-dimensional structure, leading to a drastic change in physiological activities. For instance, Shai et al. indicate that the introduction of an amphiphilic peptide having an antibacterial activity, but having strong toxicity such as hemolytic activity lost its hemolytic activity only, while retaining its antibacterial activity, and further the resulting peptide could selectively act on cancer cells only but impart less toxicity to normal cells. This indicates the possibility of developing a novel anti-tumor chemotherapy agent (Papo et al., J. Biol. Chem., 278, 210-18, 2003). This may be considered to happen that the introduction of the D-amino acid or acids probably causes a variation in a mode of action on cell membranes due to a subtle difference in the hydrophobic-hydrophilic balance, although details are not yet clarified.

The present inventors have already found that the peptide Hel 13-5 was provided with an amphiphilic property and an excellent property as a pulmonary surfactant peptide, but it demonstrated a strong hemolytic activity when used singly. Therefore, they tried to reduce the hemolytic activity of the peptide Hel 13-5 by introducing D-amino acids thereinto. The positions of the introduction of the D-amino acids were set to be balanced in an appropriate way, and the rate of introduction was designed to amount for 20% to 30% of the total number of the amino acids.

### (Conformation and Lipid-Solubility of Peptides)

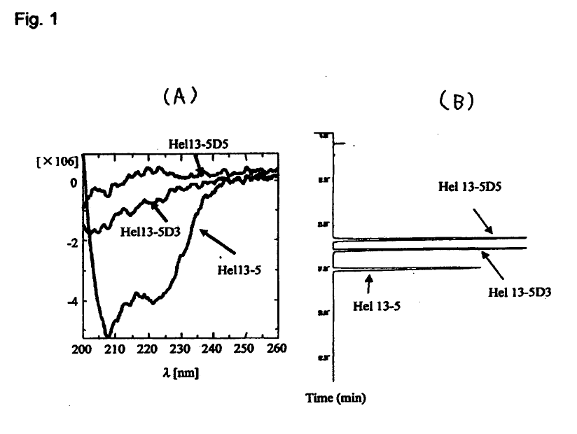

A CD spectrum measurement and a reverse-phase HPLC analysis were carried out in order to determine a change in the structure and properties of the peptides by the introduction of the D-amino acids. The results are shown in FIG. 1.

The peptides and related substances according to the present invention were measured for their CD spectra to determine their structures (see FIG. 1A). From the result of the CD spectrum measurement, it was confirmed that the peptide Hel 13-5 assumed an α-helix structure as designed, while the peptides Hel 13-5D3 and Hel 13-5D5, each with the D-amino acids introduced thereinto, reduced their α-helix structures and assumed a random-like structure. From this result, they were measured for Fourier Transform Infrared Attenuation Total Reflection method (ATR-FTIR) to determine an α-structure in addition to the helix structure (see FIG. 1B). It was further found from reverse-phase HPLC experiments that their solubility to lipids decreased as a rate of introduction of the D-amino acids increased.

From the above results, it was found that the peptides prepared so far by the present inventors, which were composed of L-amino acids only, exerted influences on their structures and solubility to lipids, as expected. These influences were considered to be derived from the side chains of the structure. In a usual case where the α-helix structure is formed only with L-amino acids, the side chains are oriented in a constant direction along the helix axis. In the case of the peptides according to the present invention in which the D-amino acids were introduced partially, the corresponding side chains may be caused to change their orientation, leading to a state in which the side chains may approximate their locations closely to one another or strike against other side chains. As a result, this may be considered to cause no retaining its original α-helix structure and lead to a decrease in the helix structure while forming a random structure including α-structure. Further, it is assumed that this may cause a partial breakage of the amphiphilic structure and a decrease in the solubility to lipids. Moreover, the measurement of fluorescence spectrum of Trp implies that these peptides are partially embedded into the lipid-soluble portion of the bimolecular lipid membrane.

### (Assessment of In Vitro Activity of Pulmonary Surfactants)

It is needed to form a stable monomolecular membrane-bimolecular membrane in order to review a mechanism of representing the activity of a pulmonary surfactant. The saturated and unsaturated phospholipids are considered as essential in terms of performing a mutual migration between the membranes. The pulmonary surfactant is composed of the protein and the lipid, and the protein is considered to act as a catalyst in order to allow the phospholipids to smoothly migrate mutually between the monomolecular membrane and bimolecular membrane at the air-liquid interfaces of the alveoli cells. This mutual migration can be easily measured by measuring a surface tension-surface area curve (a hysteresis curve) with a Wilhelmy surface tensiometer representing a variation in the respiratory pressure of the lung. Generally, it is considered that a better in vivo activity can be achieved as the speed of decreasing the surface tension at the time of compression is faster or the ability of spontaneously spreading on the surface is faster. In other words, the better activity can be achieved as the greater the area to be formed by the hysteresis curve or the smaller the surface tension at the time of compression.

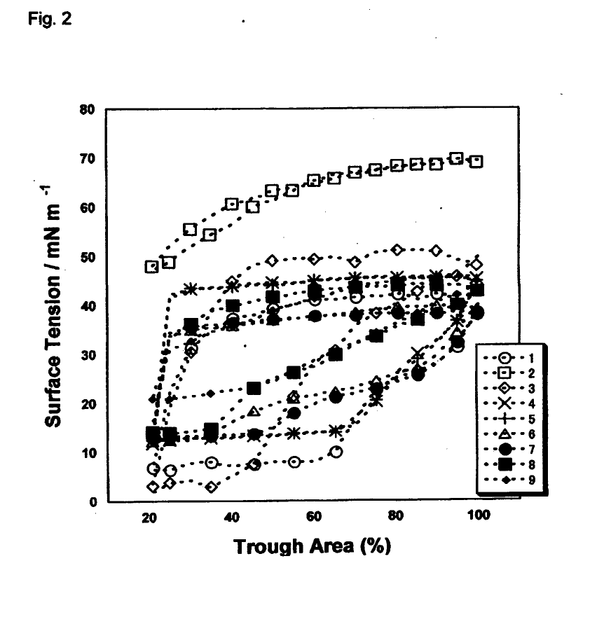

FIG. 2 shows a hysteresis curve of the D-amino acid-containing peptide-soy lecithin lipid system. In the system of Hel 13-5D3 in which three L-amino acids of Hel 13-5 are replaced by three D-amino acids, the lipid mixture system produced a favorable curve although the minimum surface tension (13 mNm⁻¹) does not exceed that of Surfacten®. On the other hand, the system of Hel 13-5D5 in which five L-amino acids of Hel 13-5 are replaced by five D-amino acids does not a better curve than the system of Hel 13-5D3.

In FIG. 2, the reference symbols are as follows:
(1): Surfacten®
(2): Exosurf
(3): Surfaxin
(4): octadecanol-egg PC-PA (40:35:25 w/w)
(5): fractionated soy lecithin 70D-hydrogenated soy lecithin-PA (40:40:20)
(6): OD-egg PC-PA-Hel 13-5 (40:35:22.5:2.5)
(7): fractionated soy lecithin 70D-hydrogenated soy lecithin-PA-Hel 13-5 (40:40:17.5:2.5)
(8): fractionated soy lecithin 70D-hydrogenated soy lecithin-PA-Hel 13-5D3 (40:40:17.5:2.5)
(9): fractionated soy lecithin 70D-hydrogenated soy lecithin-PA-Hel 13-5D5 (40:40:17.5:2.5)

### (Assessment of Activity of Pulmonary Surfactants by Animal Experiments)

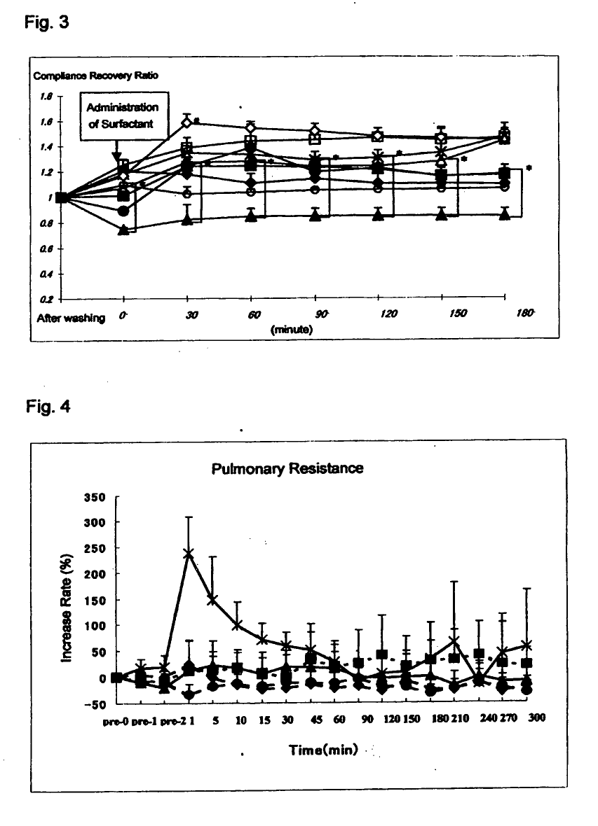

The activity of the peptide-lipid system according to the present invention was assessed on the basis of a lung compliance measured by using lung-irrigated rats as a model animal of respiratory distress syndromes causing to occur in human immature infants due to a deficiency in the pulmonary surfactant. Respiratory injury model rats were formed by irrigating the lungs of Wister rats with warm physiological saline, and the pulmonary compliance was measured by administering the rats with the pulmonary surfactant. FIG. 3 shows the results of the pulmonary compliance. In general, the compliance value arises rapidly immediately after the administration of the pulmonary surfactant, and the surfactant is considered to be better as the compliance value becomes higher. The commercial medicine Surfacten® raised its compliance value gradually and made it constant in approximately two hours after the administration of the pulmonary surfactant. On the other hand, the Hel 13-5D3-lipid mixture (i.e., fractionated soy lecithin 70D-hydrogenated soy lecithin-PA-Hel 13-5D3 (40:40:17.5:2.5) system showed tendencies similar to Surfacten®, but demonstrated rather higher values in 0.5 hour and 1 hour after administration. This indicates that this mixture system demonstrated a better recovery of the pulmonary functions than Surfacten®. The Hel 13-5D5 system, however, was found to demonstrate a recovery of the pulmonary functions weaker than the Hel 13-5D3 system. Moreover, the Hel 13-5D3 system was better than the system of a mixture of Hel 13-5 (containing no D-amino acids) with less expensive soy lipid (i.e., fractionated soy lecithin 70D-hydrogenated soy lecithin-PA-Hel 13-5). In addition, the surfactant activity of the Hel 13-5D3 system can be said to be better than that of artificial surfactant (Surfaxin) developed as a peptide-lipid system containing KL24 as a peptide.

In FIG. 3, the reference symbols are as follows:
□: Surfacten®
▴: Exosurf
: Surfaxin
♦: octadecanol-egg PC-PA (40:35:25 w/w)
▪: fractionated soy lecithin 70D-hydrogenated soy lecithin-PA (40:40:20)
Δ: OD-egg PC-PA-Hel 13-5 (40:35:22.5:2.5)
X: fractionated soy lecithin 70D-hydrogenated soy lecithin-PA-Hel 13-5 (40:40:17.5:2.5)
⋄: fractionated soy lecithin 70D-hydrogenated soy lecithin-PA-Hel 13-5D3 (40:40:17.5:2.5)
◯: fractionated soy lecithin 70D-hydrogenated soy lecithin-PA-Hel 13-5D5 (40:40:17.5:2.5)

### Example 3

### (Effects of Pulmonary Surfactants on Asthma Models)

As shown in FIG. 4, as compared with a control in which no surfactant was administered, each of the surfactant systems, i.e., fractionated soy lecithin 70D-hydrogenated soy lecithin-PA (40:40:20), fractionated soy lecithin 70D-hydrogenated soy lecithin-PA-Hel 13-5 (40:40:17.5:2.5), fractionated soy lecithin 70D-hydrogenated soy lecithin-PA-Hel 13-5D3 (40:40:17.5:2.5), and fractionated soy lecithin 70D-hydrogenated soy lecithin-PA-Hel 13-5D5 (40:40:17.5:2.5), controlled the enhancement of resistance of the airway to the egg yolk albumin (OVA) inducement in a significant way up to 45 minutes after the administration of OVA.

In FIG. 4, the reference symbols are as follows:
▴: Surfacten®
: fractionated soy lecithin 70D-hydrogenated soy lecithin-PA (40:40:20)
♦: fractionated soy lecithin 70D-hydrogenated soy lecithin-PA-Hel 13-5 (40:40:17.5:2.5)
▪: fractionated soy lecithin 70D-hydrogenated soy lecithin-PA-Hel 13-5D3 (40:40:17.5:2.5)
X: physiological saline

### INDUSTRIAL APPLICABILITY

The mixture system of the peptide, e.g., Hel 13-5D3, one of the D-amino acid-containing peptide, with soy lipid (i.e., fractionated soy lecithin 70D-hydrogenated soy lecithin-PA-Hel 13-5D3; 40:40:17.5:2.5) according to the present invention showed a favorable hysteresis curve and demonstrated a higher pulmonary surfactant activity by experiments for measuring a pulmonary compliance using lung-irrigated rats. Further, the activity of the mixture system of the present invention exceeded that of commercially available Surfacten®. Moreover, the mixture system uses lesser expensive soy lecithin in place of expensive DPPC and PG. As soy lecithin has already been used as a medicine for treating hyperlipidemia, it can be considered that the soy lecithin can eliminate problems with toxicity of the lipid in the event where it is applied to medicine. The hemolytic activity of the peptide Hel 13-5D3 itself is lower by several times to the peptide Hel 13-5, and the toxicity can be said to be improved in this respect. Moreover, the peptide Hel 13-5, when applied in the form of the lipid mixture system, does not demonstrate a hemolytic property. In addition, commercially available artificial pulmonary surfactant derived from an animal lung may always be encountered with the risk of BSE. On the contrary, the pulmonary surfactant composition of the present invention does not have such a concern because it does not use any ingredient derived from an animal lung and further it can be stably supplied as a material for a medicine for treating diseases such as severe respiratory disorders, e.g., infant's respiratory distress syndromes (RDS).

On the other hand, as the peptide containing D-amino acids according to the present invention can be synthesized on a large scale by usual peptide synthesis methods, it can be prepared from simple amino acids as a raw material so that it can be expected to be practically applicable.

As the peptide having such a useful and high surfactant activity can be used without any concern about infection with infectious bacteria or with an extreme safety because it does not contain any animal-derived protein, it can be prepared at reasonable costs so that it can be expected to be applicable to acute respiratory distress syndromes (ARDS) occurring in adult people to whom it has not been applied so far due to its too expensive costs.

There is also a report of the effects on the control of a fit of asthma of some artificial surfactants including Surfacten®. This report, however, was made simply on the result of experiments conducted on a small scale because cost performances and mechanisms were not established at that time. On the contrary, our results of the experiments confirm the effects on a control over a fit of asthma of the peptide-lipid mixture systems, i.e., fractionated soy lecithin 70D-hydrogenated soy lecithin-PA, fractionated soy lecithin 70D-hydrogenated soy lecithin-PA-Hel 13-5, and fractionated soy lecithin 70D-hydrogenated soy lecithin-PA-Hel 13-5D3, like Surfacten®. It can be noted, however, that the peptide-lipid mixture systems according to the present invention can demonstrate the merit that they can be prepared at more inexpensive costs, as compared with conventional surfactants, and they can be considered to be clinically developed as an asthma curing agent. Further, it is very interesting from the point of view of clarifying a mechanism of the effect of the surfactants on a control over a fit of asthma that the fractionated soy lecithin 70D-hydrogenated soy lecithin-PA system, which does not contain any surfactant protein, exhibited the same effects as the conventional ones.

The pulmonary surfactant composition according to the present invention, which does not depend upon the bovine lung, indicates the possibility of application to asthma, mediation of severe respiratory distress at the terminal stage such as lung cancer and so on, and other severe cases of various respiratory diseases including pneumonia and so on, in addition to application to RDS and ADRS.

1-18. (canceled)

19. An amphiphilic peptide, which is composed of a hydrophilic portion and a hydrophobic portion, has specificity to a mode of action against membrane and which is provided with a surfactant activity, but which has no or little hemolytic activity, comprising an amino acid sequence of 5 to 60 amino acids in which 5% to 50% of the amino acid sequence is replaced by D-amino acid or acids.

20. The peptide as claimed in claim 19, wherein 10% to 40% of said amino acid sequence is replaced with the D-amino acid or acids.

21. The peptide as claimed in claim 19, wherein said amino acid sequence contains from one amino acid to ten D-amino acids.

22. The peptide as claimed in claim 19, wherein said amino acid sequence comprises leucine and/or lysine as a major structuring amino acid and, as needed, tryptophan.

23. A pulmonary surfactant composition comprising a mixture system of a peptide as claimed in claim 19 and a natural lecithin at a rate of from approximately 2 to 8 to approximately 8 to 2.

24. The pulmonary surfactant composition as claimed in claim 23, further comprising a fatty acid and/or a higher alcohol.

25. The pulmonary surfactant composition as claimed in claim 24, wherein said fatty acid comprises palmitic acid and said higher alcohol comprises octadecanol.

26. A method of use comprising applying the peptide as claimed in claim 19 as a pulmonary surfactant to respiratory disorders.

27. The method of use as claimed in claim 26, wherein said respiratory disorder infant's respiratory distress syndrome or acute respiratory distress syndrome.
